(19) United States
(12) Patent Application Publication
(10) Pub. No.: US 2024/0358270 A1
(43) Pub. Date: Oct. 31, 2024

(54) Patient Positioning Using a Patient Table Movable Within a Patient Receiving Area

(71) Applicant: Siemens Healthineers AG, Forchheim (DE)

(72) Inventors: Bernhard Krauss, Burgthann (DE); Martino Leghissa, Wiesenthau (DE); Matthias Malzacher, Nürnberg (DE)

(73) Assignee: Siemens Healthineers AG, Forchheim (DE)

(21) Appl. No.: 18/645,676

(22) Filed: Apr. 25, 2024

(30) Foreign Application Priority Data
Apr. 25, 2023 (DE) 10 2023 203 811.0

(51) Int. Cl. A61B 5/055; A61B 5/00

(52) U.S. Cl. CPC A61B 5/055; A61B 5/704

(57) ABSTRACT

A method for patient table positioning using a patient table of a magnetic resonance device that is movable within a patient receiving area, wherein the patient table has at least two degrees of freedom with respect to its movement within the patient receiving area. Initially, the patient table is positioned within the patient receiving area in a first patient table position and first magnetic resonance data from the patient is captured. The first magnetic resonance data is used to determine a position of a region of interest and to ascertain a second patient table position, wherein in the second patient table position, the region of interest has a minimum possible distance from an isocenter. This is followed by positioning in the second patient table position, wherein at least two degrees of freedom are available to the patient table for movement into the at least one second patient table position.

### TECHNICAL FIELD

The present disclosure relates to a method for patient positioning using a patient table that can be moved within a patient receiving area, wherein the patient table has at least two degrees of freedom with respect to its movement within the patient receiving area. Furthermore, the present disclosure relates to a magnetic resonance device embodied to execute the method for patient positioning using a patient table that can be moved within a patient receiving area. In addition, the disclosure is furthermore based on a computer program product embodied to execute the method for patient positioning using a patient table that can be moved within a patient receiving area.

### BACKGROUND

Irrespective of the grammatical gender of specific terms, they also include individuals with male, female, or other gender identities.

Previous magnetic resonance examinations entailed positioning a target anatomy and/or a region of interest of the patient in only one single direction within the patient receiving area, in particular, a cylindrical patient receiving area. This direction is the z- direction of the magnetic resonance device and/or the longitudinal direction of the patient receiving area. However, depending, for example, on the region of interest of the patient, for example, an elbow region, a hip region, and/or a hand region etc., it may be desirable to position the patient and thus move the patient table with a further degree of freedom, such as, for example, in the transverse direction of the patient receiving area. Furthermore, depending on a patient's anatomy, positioning, and/or movement within the patient receiving area with at least one further degree of freedom is also desirable. For example, in the case of obese patients, the same organ regions are arranged in different positions on the patient table in the x-direction and/or y-direction compared to the case with small children.

Magnetic resonance devices now also have patient receiving areas, in particular cylindrical patient receiving areas, with a large diameter of, for example, 70 cm, 75 cm, 80 cm, or more. Herein, this type of patient receiving area provides enough space to allow the patient table to be displaced and/or moved with several degrees of freedom. On the other hand, an imaging volume and/or a field of view (FOV) within the patient receiving area is restricted and does not extend over the entire available width and/or height, so that displacement and/or movement in at least two degrees of freedom of the patient table is advantageous.

### SUMMARY

Therefore, an object underlying the present disclosure is, in particular, to enable simple and quick positioning of the patient table in at least two degrees of freedom. The object is achieved by the features of the claims.

The disclosure is based on a method for patient table positioning by means of a patient table of a magnetic resonance device that can be moved within a patient receiving area, wherein the patient table has at least two degrees of freedom with respect to its movement within the patient receiving area, comprising the following method steps:
introducing a patient into the patient receiving area, wherein, for this purpose, the patient table is positioned in a first patient table position within the patient receiving area,
capturing first magnetic resonance data from the patient, in particular a region of interest of the patient,

determining first image data from the first magnetic resonance data, wherein at least one target anatomy of the patient and/or a position of a region of interest with the at least one target anatomy is ascertained and/or determined in the first image data,
ascertaining at least one second patient table position, wherein, in the at least one second patient table position, the at least one region of interest and/or at least one target anatomy of the patient has a minimum possible distance from an isocenter of the magnetic resonance device, and
positioning the patient table in the at least one second patient table position, wherein at least two degrees of freedom are available to the patient table for movement into the at least one second patient table position.

The positioning of the patient table within the patient receiving area preferably takes place in an automated and/or autonomous manner. For automated and/or autonomous patient table positioning, the magnetic resonance device has a control unit embodied to control the automated and/or autonomous patient table positioning. The control unit comprises at least one computing module and/or a processor. For example, the control unit is, in particular, embodied to execute computer-readable instructions. In particular, the control unit comprises a memory unit, wherein computer-readable information is stored on the memory unit, wherein the control unit is embodied to load the computer-readable information from the memory unit and execute the computer-readable information. In this way, the control unit is embodied to execute a method for automated patient positioning by means of a patient table that can be moved in at least two spatial directions within a patient receiving area of a magnetic resonance device, together with the magnet unit and the patient support device.

The components of the control unit can be predominantly embodied in the form of software components. However, in principle, in particular, where particularly fast calculations are involved, these components can also be partially implemented in the form of software-supported hardware components, for example, FPGAs or the like. The interfaces required can likewise be embodied as software interfaces, for example, if it is only a matter of transferring data from other software components. However, they can also be embodied as interfaces constructed as hardware that are actuated by suitable software. Of course, it is also conceivable for a plurality of said components to be implemented combined in the form of a single software component or software-supported hardware component.

To be moved into the patient receiving area, the patient is already positioned on the patient table. In addition, all accessory units required for the upcoming magnetic resonance examination are already located on the patient and/or on the patient table in a correct position for the upcoming magnetic resonance examination. Such an accessory unit can, for example, comprise a local radio-frequency coil and/or an ECG unit and/or supporting aids, etc. In this first patient table position, the patient table is preferably positioned in such a way within the patient receiving area that a target anatomy of the patient and/or region of interest of the patient is arranged within the FOV. In addition, positioning in the first patient table position can take place with the aid of markers and/or with the aid of the localization of a local radio-frequency coil and/or user input, etc.

The first magnetic resonance data can, for example, be captured by means of a localizer scan by a magnet unit of the magnetic resonance device. This first magnetic resonance data is not used for diagnostic and/or medical evaluation. This first magnetic resonance data preferably only comprises a few slices for ascertaining a target anatomy and/or the region of interest of the patient. In addition, the first magnetic resonance data can comprise data, in particular slices, with different orientations and/or data from different positions.

The first magnetic resonance data is preferably evaluated by means of the control unit, in particular in an automated manner by means of the control unit, which, for this purpose, in particular comprises an evaluation unit. In particular, the first magnetic resonance data is used to generate image data, and a target anatomy of the patient and/or a position of a region of interest is ascertained and/or determined in this image data. The target anatomy preferably comprises the organ and/or the body region of the patient for which a clinical and/or diagnostic issue exists and/or which is to be clarified. In addition, it is also possible for a plurality of target anatomies to be determined in the first image data, for example, left hip and right hip. The region of interest comprises the region in which medical and/or diagnostic magnetic resonance data is to be captured. Herein, the region of interest of the patient comprises the at least one target anatomy. In the case of a plurality of target anatomies, it is also possible for a plurality of regions of interest to be defined.

The first magnetic resonance data or the target anatomy determined from the first magnetic resonance data and/or region of interest is used to ascertain the second patient table position. In the second patient table position, the at least one target anatomy and/or a center of the region of interest has a minimum distance from the isocenter. Herein, preferably a center and/or a middle of the target anatomy and/or region of interest has a minimum possible distance from the isocenter of the magnetic resonance device, in particular the magnet unit. Herein, the minimum possible distance may be dependent on the space available for the movement of the patient table within the patient receiving area. For example, if only limited space is available in the transverse direction of the patient receiving area for positioning the patient table, the region of interest in the transverse direction is not always positioned in the isocenter for a magnetic resonance examination. If there is a plurality of target anatomies and/or a plurality of regions of interest, it is also possible to ascertain two or more second patient table positions.

For moving the patient table into the second patient table position, at least two degrees of freedom and preferably four degrees of freedom are available to the patient table. However, the movement of the patient table into the second patient table position does not necessarily comprise a movement with four degrees of freedom and instead can also comprise fewer than four degrees of freedom. Herein, the patient table can be positioned in the second patient table position automatically and/or autonomously by means of the control unit. Alternatively, the patient table can also be positioned in the second patient table position manually by an operator.

The magnetic resonance device preferably comprises a medical and/or diagnostic magnetic resonance device configured and/or embodied to capture medical and/or diagnostic image data, in particular medical and/or diagnostic magnetic resonance image data, from a patient. The magnetic resonance device comprises a magnet unit with a main magnet, a gradient coil unit, and a radio-frequency antenna unit.

The main magnet is embodied to generate a homogeneous main magnetic field with a defined magnetic field strength, such as, for example, a magnetic field strength of 0.55 T, 1.5 T, 3 T, 7 T, etc. In particular, the main magnet is embodied to generate a strong, constant, and homogeneous main magnetic field. The homogeneous main magnetic field is preferably arranged or located within a patient receiving area of the magnetic resonance device.

For a magnetic resonance examination, the patient, in particular the region of interest of the patient, is positioned within the patient receiving area of the magnetic resonance device. Herein, the patient receiving area is at least partially surrounded by the magnet unit, in particular, surrounded by the magnet unit in a cylindrical shape. Preferably, a field of view (FOV) and an isocenter of the magnetic resonance device are arranged within the patient receiving area. The FOV preferably comprises a capturing region of the magnetic resonance device within which the conditions for capturing medical image data, in particular magnetic resonance image data within the patient receiving area, are present, such as, for example, a homogeneous main magnetic field. The isocenter of the magnetic resonance device preferably comprises the region and/or point within the magnetic resonance device that has the optimum and/or ideal conditions for capturing medical image data, in particular, magnetic resonance image data. In particular, the isocenter comprises the most homogeneous magnetic field region within the magnetic resonance device.

For moving the patient in and/or positioning the patient, in particular the region of interest of the patient, within the patient receiving area, the magnetic resonance device comprises a patient support device. Herein, the patient support device has a patient table embodied to be moved into the patient receiving area. In addition to the direction of entry, in particular, the z-direction, the patient table has at least one further degree of freedom with respect to its movement within the patient receiving area. Herein, preferably, the patient table has three degrees of freedom with respect to its movement within the patient receiving area. Herein, particularly advantageously, the patient table has at least four degrees of freedom with respect to its movement within the patient receiving area. Preferably, a first degree of freedom comprises a movement in the z-direction. Preferably, a second degree of freedom comprises a movement in the x-direction. Preferably, a third degree of freedom comprises a movement in the y-direction. Preferably, a fourth degree of freedom comprises a rotational movement about a vertical axis. Herein, the vertical axis for the rotational movement can run through the isocenter of the magnet unit. In addition, the patient table can comprise further degrees of freedom of movement.

The aspect according to the disclosure enables simple and quick positioning of the patient table, and thus of the target anatomy and/or region of interest, to be achieved for a magnetic resonance examination. Furthermore, this also enables body parts on the side of the patient, for example arms, to be positioned as close as possible to the isocenter for a magnetic resonance examination, without this requiring the patient having to be moved. In particular, this enables high image quality to be achieved in captured magnetic resonance data since the target anatomy and/or region of interest has a minimum distance from the isocenter. In addition, rotating the patient table about a vertical axis can also provide a medical operator, for example, a physician, with easy access to the patient, for example, for a medical procedure or medical intervention during a magnetic resonance examination.

In an advantageous development of the method according to the disclosure, it can be provided that, after positioning the patient table in the at least one second patient table position, at least one parameter of the region of interest is adjusted to the at least one second patient table position. The adjustment of the at least one parameter of the region of interest preferably comprises automatic and/or autonomous adjustment of the at least one parameter. In particular, herein, position parameters of the region of interest are adjusted to the changed patient table position, in particular, the second patient table position. This makes it possible to ensure that correct position data for the region of interest is always available for the upcoming magnetic resonance examination.

In an advantageous development of the method according to the disclosure, it can be provided that, after positioning the patient table in the at least one second patient table position, second magnetic resonance data is captured. The second magnetic resonance data preferably comprises medical and/or diagnostic magnetic resonance data. In particular, the second magnetic resonance data is consulted and/or used to clarify a medical and/or diagnostic issue. Herein, the second magnetic resonance data preferably has a higher resolution than the first magnetic resonance data. This aspect of the disclosure has the advantage that high image quality can be achieved in captured magnetic resonance data, in particular the second magnetic resonance data. In particular, herein, distortion and/or image artifacts in the second magnetic resonance data can be reduced and/or prevented.

In an advantageous development of the method according to the disclosure, it can be provided that a collision model is used to ascertain the at least one second patient table position. The collision model preferably comprises information about the size of the patient and/or the patient table. Herein, in addition to information about the size of the patient, the collision model can also include a safety value, in particular, a safety distance of the patient from the enclosure surrounding the patient receiving area. Preferably, the collision model enables the second patient table position to be determined in such a way that neither the patient nor the patient table collides with and/or comes into contact with the enclosure surrounding the patient receiving area in the second patient table position. This enables a high degree of safety to be provided for the patient during the magnetic resonance examination. For example, this enables undesirable contact of the patient with the enclosure surrounding the patient receiving area to be prevented. Furthermore, this also advantageously prevents lines and/or cables from being trapped between the patient table and the enclosure surrounding the patient receiving area during movement of the patient table.

In an advantageous development of the method according to the disclosure, it can be provided that the collision model comprises an anatomy and/or size of the patient to be examined and/or position information for the patient table. Herein, the collision model can also comprise a patient model, which is determined on the basis of the anatomy and/or size of the patient. Such a patient model can, for example, be ascertained based on the first magnetic resonance data and/or based on camera data. For example, a camera can be used to capture a size and/or extent of the patient before the patient table is moved into the patient receiving area. In addition, such a patient model can also already be stored in a database.

Herein, the position information for the patient table can also comprise accessory units that are arranged on the patient table to support the patient, such as, for example, additional arm rests etc., that protrude beyond a table top of the patient table. To determine and/or capture the position information for the patient table, the patient table can be provided with markers, in particular MR-visible markers, so that the position information is available and/or can be ascertained from the first magnetic resonance data. In addition, the position information for the patient table can also be captured by means of a camera, for example, before the patient table is moved into the patient receiving area.

Here, once again, a high level of safety can be provided for the patient during the magnetic resonance examination and an undesired collision of the patient with the enclosure surrounding the patient receiving area can be prevented. Furthermore, this can also advantageously prevent lines and/or cables from becoming trapped between the patient table and the enclosure surrounding the patient receiving area during a movement of the patient table.

In an advantageous development of the method according to the disclosure, it can be provided that, after positioning of the patient table in the at least one second patient table position, at least one adjustment step is performed. Such an adjustment step can comprise checking adjustment settings as to whether they require adjustment for the second patient table position. For example, here, shim settings can be adjusted in order to obtain high image quality in the captured second magnetic resonance data.

In an advantageous development of the method according to the disclosure, it can be provided that, if there are two or more target anatomies of the patient, and thus two or more regions of interest, a second patient table position is ascertained and/or determined for each of the regions of interest. This advantageously enables simple and quick positioning of the patient table to be achieved for each of the regions of interest. In particular, an ideal examination position for the patient can be ascertained for each target anatomy and/or each region of interest.

In an advantageous development of the method according to the disclosure, it can be provided that the capturing of the first magnetic resonance data is repeated if not all target anatomies and/or regions of interest of the patient are captured and/or determined during a first capturing of first magnetic resonance data. This enables a plurality of second patient table positions for medical and/or diagnostic magnetic resonance measurements to be ascertained and also for optimized movement of the patient table to the different second patient table positions to be achieved.

In an advantageous development of the method according to the disclosure, it can be provided that, for ascertaining a plurality of second patient table positions, in each case the preceding second patient table position is taken into account as the starting point for a patient table movement. This enables quick positioning of the patient table for different examination positions.

In an advantageous development of the method according to the disclosure, it can be provided that the capturing of the first magnetic resonance data is performed with distortion correction. Distortion artifacts can, for example, occur due to non-linearity of the magnetic field gradients, wherein such distortion artifacts can occur to a greater extent with increasing distance from the isocenter of the magnet unit. This can, in particular, occur with the first magnetic resonance data, since here, the target anatomy and/or region of interest can also be arranged in a peripheral region of the FOV. Distortion correction enables these effects to be counteracted so that the first magnetic resonance data enables an unambiguous and reliable determination of the target anatomies and regions of interest.

Furthermore, the disclosure is based on a magnetic resonance device with a magnet unit, a patient receiving area at least partially surrounded by the magnet unit, a patient table that can be moved within the patient receiving area, wherein the patient table has at least two degrees of freedom with respect to its movement within the patient receiving area, and a control unit, wherein the control unit is embodied to control the magnetic resonance device in such a way that a method for automated patient positioning by means of a patient table that can be moved within a patient receiving area is executed.

The magnetic resonance device according to the disclosure enables simple and quick positioning of the patient table and thus of the target anatomy and/or region of interest for a magnetic resonance examination to be achieved. In particular, this enables high image quality to be achieved in captured magnetic resonance data since the target anatomy and/or region of interest has a minimum distance from the isocenter.

The advantages of the magnetic resonance device according to the disclosure substantially correspond to the advantages of the method according to the disclosure for patient positioning by means of a patient table that can be moved within a patient receiving area, which are described in detail above. Features, advantages, or alternative aspects mentioned here can likewise be transferred to the other claimed subject matter and vice versa.

In an advantageous development of the magnetic resonance device according to the disclosure, it can be provided that the patient table has four degrees of freedom for movement within the patient receiving area. Preferably, a first degree of freedom comprises movement in the z-direction, a second degree of freedom comprises movement in the x-direction, a third degree of freedom comprises movement in the y-direction, and a fourth degree of freedom comprises rotational movement about a vertical axis. Herein, the vertical axis for the rotational movement can run through the isocenter of the magnet unit. The four degrees of freedom enable the position of the patient table to be adjusted individually to the patient and/or an examination. In particular, rotating the patient table about a vertical axis can additionally provide a medical operator, for example, a physician, with easy access to the patient, for example, for a medical procedure and/or medical intervention during a magnetic resonance examination.

Furthermore, the disclosure is based on a computer program product, which comprises a program and can be loaded directly into a memory of a programmable control unit, with program means for controlling a method for patient positioning by means of a patient table of a magnetic resonance device that can be moved within a patient receiving area, wherein the patient table has at least two degrees of freedom with respect to its movement within the patient receiving area when the program is executed in the control unit. Herein, the computer program may require program means, for example, libraries and auxiliary functions, in order to implement the corresponding aspects of the method. Herein, the computer program can comprise software with a source code, which still has to be compiled and linked or only needs to be interpreted, or an executable software code that only needs to be loaded into a corresponding computing unit for execution.

The computer program product according to the disclosure can be loaded directly into a memory of a programmable control unit and/or computing unit and has program code means for executing a method according to the disclosure when the computer program product is executed in the control unit and/or computing unit. The computer program product can be a computer program or comprise a computer program. This enables the method according to the disclosure to be executed quickly, identically, repeatedly, and robustly. The computer program product is configured in such a way that it can execute the method steps according to the disclosure by means of the control unit and/or computing unit. Herein, the control unit and/or computing unit must in each case fulfill the requisite conditions, such as, for example, having a corresponding random-access memory, a corresponding graphics card or a corresponding logic unit, so that the respective method steps can be executed efficiently. The computer program product is, for example, stored on a computer-readable medium or held on a network or server from where it can be loaded into the processor of a local computing unit, which can be directly connected to the magnetic resonance device or embodied as part of the magnetic resonance device. Furthermore, control information of the computer program product can be stored on an electronically readable data carrier. The control information of the electronically readable data carrier can be embodied to execute a method according to the disclosure when the data carrier is used in a computing unit. Thus, the computer program product can also represent the electronically readable data carrier. Examples of electronically readable data carriers are DVDs, magnetic tapes, hard disks, or USB sticks on which electronically readable control information, in particular software (see above), is stored.

### BRIEF DESCRIPTION OF THE DRAWINGS

Further advantages, features, and details of the disclosure emerge from the exemplary aspect described in the following and from the drawings.

FIG. 1 illustrates a schematic representation of a magnetic resonance device according to the disclosure,

FIG. 2 illustrates a method according to the disclosure for patient positioning by means of a patient table in at least two spatial directions within a patient receiving area,

FIG. 3 illustrates a first example of a first patient table position of a patient,

FIG. 4 illustrates a second example of a first patient table position of a second patient,

FIG. 5 illustrates the first example with a second patient table position, and

FIG. 6 illustrates the second example with a second patient table position.

### DETAILED DESCRIPTION

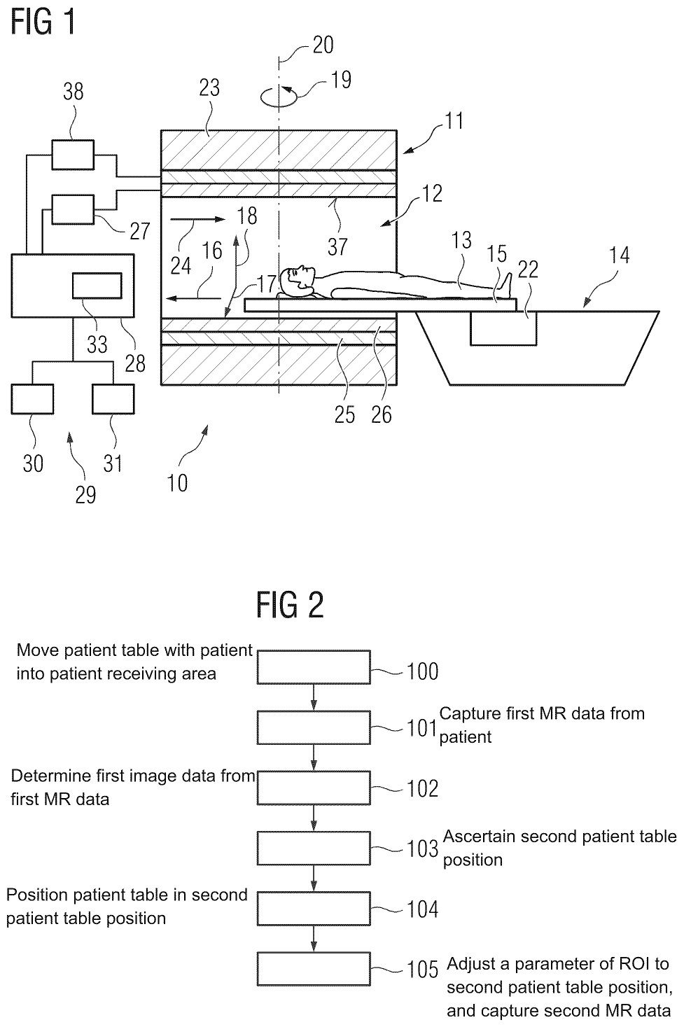

FIG. 1 is a schematic representation of a magnetic resonance device 10. The magnetic resonance device 10 comprises a magnet unit 11. In addition, the magnetic resonance device 10 has a patient receiving area 12 for receiving a patient 13. In the present exemplary aspect, the patient receiving area 12 is cylindrical in shape and is surrounded by the magnet unit 11 in a cylindrical shape in a circumferential direction. However, in principle, a different aspect of the patient receiving area 12 is conceivable at any time.

The patient 13 is pushed and/or moved into the patient receiving area 12 for a magnetic resonance examination by means of a patient support device 14 of the magnetic resonance device 10. For this purpose, the patient support device 14 has a patient table 15 embodied to be movable within the patient receiving area 12. The patient table 15 has four degrees of freedom 16, 17, 18, 19 for movement within the patient receiving area 12. A first degree of freedom 16 comprises a movement in the longitudinal direction or z-direction of the patient receiving area 12. A second degree of freedom 17 comprises a movement in the transverse direction or x-direction of the patient receiving area 12. A third degree of freedom 18 comprises a movement in the vertical direction or y-direction of the patient receiving area 12. A fourth degree of freedom 19 comprises a rotary movement and/or rotational movement about a vertical axis 20 of the patient receiving area 12. For the rotary movement and/or rotational movement, the vertical axis 20 preferably runs through the isocenter 21 of the magnet unit 11. For this purpose, the patient support device 14 has a setting unit 22 with a drive unit. The drive unit generates a driving torque, which is transmitted by means of the setting unit 22 into a movement of the patient table 15 in at least one of the four degrees of freedom 16, 17, 18, 19.

The magnet unit 11 comprises, for example, a superconducting main magnet 23 for generating a strong and, in particular, constant main magnetic field 24. Furthermore, the magnet unit 11 has a gradient coil unit 25 for generating magnetic field gradients used for spatial encoding during imaging. The gradient coil unit 25 is controlled by means of a gradient control unit 38 of the magnetic resonance device 10. The magnet unit 11 furthermore comprises a radio-frequency antenna unit 26 for exciting polarization that is established in the main magnetic field 24 generated by the main magnet 23. The radio- frequency antenna unit 26 is controlled by a radio-frequency antenna control unit 27 of the magnetic resonance device 10 and emits radio-frequency magnetic resonance sequences into the patient receiving area 12 of the magnetic resonance device 10.

To control the main magnet 23, the gradient control unit 38 and to control the radio-frequency antenna control unit 27, the magnetic resonance device 10 has a system control unit 28. The system control unit 28 controls the magnetic resonance device 10 centrally, such as, for example, by performing a predetermined imaging gradient echo sequence. In addition, the system control unit 28 comprises an evaluation unit (not shown in detail) for evaluating medical image data captured during the magnetic resonance examination.

Furthermore, the magnetic resonance device 10 comprises a user interface 29 connected to the system control unit 28. Control information, such as, for example, imaging parameters and reconstructed magnetic resonance images, can be displayed on a display unit 30, for example on at least one monitor, of the user interface 29 for a medical operator. Furthermore, the user interface 29 has an input unit 31 by means of which information and/or parameters can be input by the medical operator during a measurement process.

The magnetic resonance device 10 shown can, of course, comprise further components that magnetic resonance devices 10 typically have. In addition, the general mode of operation of a magnetic resonance device 10 is known to the person skilled in the art so that a detailed description of the further components will be dispensed with.

FIG. 2 shows a representation of a method according to the disclosure for patient table positioning by means of the patient table 15 that can be moved within the patient receiving area 12. At the start of the method, the patient 13 is already positioned on the patient table 15. In addition, all accessory units 32 required for the upcoming magnetic resonance examination, such as, for example, local radio-frequency coils and/or supporting aids etc., are also positioned on the patient 13 and/or on the patient table 15. The method for patient table positioning is controlled by means of a control unit 33 of the magnetic resonance device 10. Herein the control unit 33 can be comprised by the system control unit 28. In addition, the control unit 33 can also comprise a unit independent of the system control unit 28.

To control the method for patient table positioning, in particular automated patient table positioning, the control unit 33 comprises corresponding control software and/or control programs, which are stored in a memory unit of the control unit 33. Herein, to control the method, the control software and/or the control programs are executed by a processor of the control unit 33. Here, the control unit 33 generates control signals that are transmitted to the setting unit 22 and/or to the magnet unit 11.

In a first method step 100, the patient table 15 is moved together with the patient 13 into the patient receiving area 12. Here, the patient table 15 is positioned in a first patient table position within the patient receiving area 12. The moving-in process is triggered manually by the user by informing the system, in particular the control unit 33, by means of the user interface 29 that the preparation of the patient 13 has been completed and the patient table 15 is ready to be moved in. The positioning of the patient table 15 in this first patient table position preferably takes place in an automated manner by means of the setting unit 22, wherein here the positioning is controlled by the control unit 28. This first patient table position includes a region of interest 34 of the patient 13 within a FOV of the magnet unit 11.

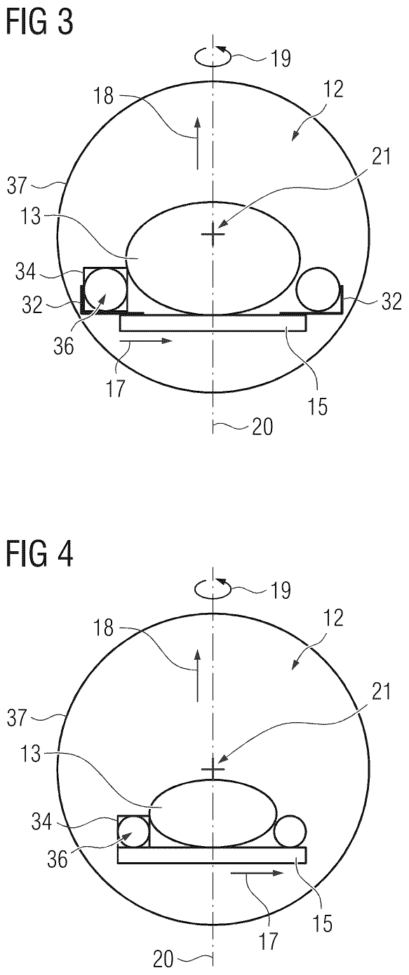

FIGS. 3 and 4 in each case show an example of the positioning of a patient 13 in a first patient table position, wherein FIGS. 3 and 4 represent a view in an xy plane of the patient receiving area 12. Both examples include the same target anatomy 36, namely the left arm of the respective patient 13, for a magnetic resonance examination. However, the two patients 13 have different anatomies, in particular sizes, so that the target anatomy 36 is in each case positioned at different positions with respect to the isocenter 21 of the magnet unit 11.

In a second method step 101, first magnetic resonance data is captured from the patient 13. The first magnetic resonance data is captured by means of the magnet unit 11, controlled by the control unit 28. In particular, herein, the first magnetic resonance data maps a region of the patient comprising the target anatomy 36 and/or region of interest 34 of the patient 13. Herein, the first magnetic resonance data can comprise data with different orientations and/or slices with different orientations and/or positions. In addition, the capturing of the first magnetic resonance data can comprise distortion correction.

In a third method step 102, first image data is determined from the first magnetic resonance data by means of the control unit 28, wherein the determination of the first image data from the first magnetic resonance data takes place automatically and/or autonomously by means of the control unit 28. Then, a target anatomy 36 and/or a position of the region of interest 34 of the patient 13 is determined in the first image data, see FIGS. 3 and 4.

In a fourth method step 103, a second patient table position is ascertained, wherein, in the second patient table position, the region of interest 34 and/or target anatomy 36 of the patient 13 has a minimum possible distance from the isocenter 21 of the magnetic resonance device 10, in particular the magnet unit 11. The second patient table position is ascertained automatically and/or autonomously by means of the control unit 28. In particular, a center of the target anatomy 34 and/or region of interest 34 of the patient 13 comprises a minimum distance from the isocenter 21 of the magnet unit 11.

Herein, the second patient table position is ascertained using a collision model, wherein the collision model comprises an anatomy and/or size of the patient 13 and/or position information for the patient table 15. Herein, the collision model can also comprise a patient model determined based on the anatomy and/or size of the patient 13. Such a patient model can, for example, be ascertained using the first magnetic resonance data and/or camera data. In addition, such a patient model can also already be stored in a database. Herein, the position information for the patient table 15 can also comprise accessory units 32 arranged on the patient table 15 for supporting the patient 13, such as, for example, additional arm rests and/or supporting elements, etc., which protrude beyond a table top of the patient table 15. To determine and/or capture the position information for the patient table 15, the position information can also be provided with markers, in particular MR-visible markers, so that the position information can be ascertained from the first magnetic resonance data. In addition, the position information for the patient table 15 can also be captured by means of a camera. Preferably, the collision model determines the second patient table position in such a way that neither the patient 13 nor the patient table 15 collides with and/or comes into contact with an enclosure 37 surrounding the patient receiving area 12.

Then, in a fifth method step 104, the patient table 15 is positioned in the second patient table position. The patient table 15 is positioned in this second patient table position in an automated manner and/or autonomously by means of the setting unit 22 and controlled by the control unit 28. For positioning in this second patient table position, the patient table 15 can execute a movement in the four degrees of freedom 16, 17, 18, 19.

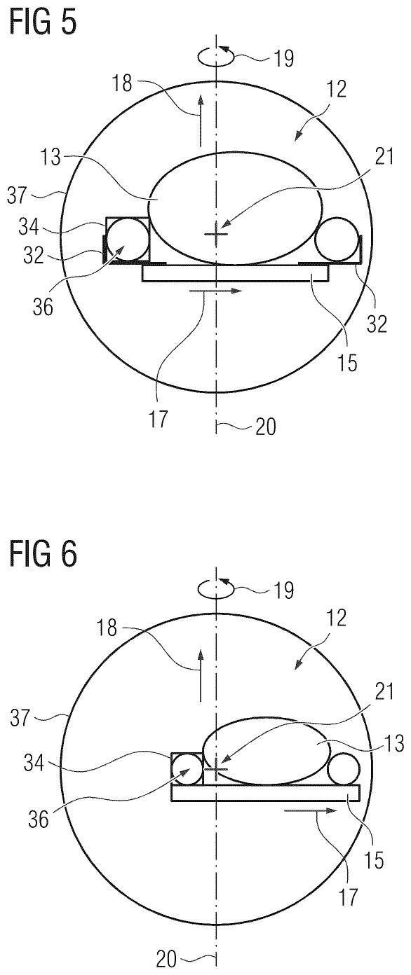

FIGS. 5 and 6, in each case, show the positioning of the patient table 15 in the second patient table positions for the two examples in FIGS. 3 and 4. Herein, in the case of the same target anatomy 36, for example, an arm of the patient 13, there may be a different distance between the target anatomy 36 and/or region of interest 34 and the isocenter 21 of the magnet unit 11. For example, in the case of a large and/or obese patient 15 (FIG. 5), there is a greater distance between the target anatomy 36, in particular, the arm and/or region of interest 34 and the isocenter 21 of the magnet unit 11 than there is with a thin patient 15 (FIG. 6).

Then, in a sixth method step 105, at least one parameter of the region of interest 34 is adjusted to the at least one second patient table position and then second magnetic resonance data is captured. This second magnetic resonance data comprises medical and/or diagnostic magnetic resonance data, which is intended to clarify a medical and/or diagnostic issue for the patient 13. Herein, it can also be provided in this sixth method step 105 that additional adjustment steps are performed before the second magnetic resonance data is captured. Such adjustment steps can comprise checking adjustment settings as to whether they require adjustment for the second patient table position. For example, here, shim settings can be adjusted in order to obtain high image quality in the captured second magnetic resonance data.

If there is a plurality of target anatomies 36 of a patient 13 and thus also a plurality of regions of interest 34 for which a magnetic resonance measurement is to be performed in each case, a second patient table position is ascertained for each of this plurality of target anatomies 36 and/or for each of the regions of interest 34 in which the respective region of interest 34 has a minimum distance from the isocenter 21 of the magnet unit 11. Herein, it may be the case that first magnetic resonance data is captured only once and the plurality of target anatomies 36 are mapped in this first magnetic resonance data. For example, when examining the left shoulder and the right shoulder, both shoulder regions of a patient 13 can be mapped in the first magnetic resonance data. On the other hand, if the plurality of target anatomies 36 are further apart and are not captured in a first capturing of first magnetic resonance data, the method steps 100 to 104 are repeated in order to obtain first magnetic resonance data for all target anatomies 36 and to ascertain and/or determine a second patient table position for the respective target anatomy 36. Herein, it may be the case that, for the ascertaining of the second patient table positions, in each case, the preceding second patient table position is taken into account as a starting point for a table movement of the patient table 15.

Although the aspects of the disclosure have been described in more detail by the preferred exemplary aspect, the disclosure is not restricted by the disclosed examples, and other variations can be derived herefrom by the person skilled in the art without departing from the scope of protection of the aspects of the disclosure.

1. A method for patient positioning using a patient table of a magnetic resonance device that can be moved within a patient receiving area, wherein the patient table has at least two degrees of freedom with respect to its movement within the patient receiving area, the method comprising:
introducing a patient into the patient receiving area, wherein, for this purpose, the patient table is positioned in a first patient table position within the patient receiving area;
capturing first magnetic resonance data from the patient;
determining first image data from the first magnetic resonance data, wherein at least one target anatomy of the patient and/or a position of at least one region of interest with the at least one target anatomy is ascertained and/or determined in the first image data;
ascertaining at least one second patient table position, wherein, in the at least one second patient table position, the at least one region of interest and/or at least one target anatomy of the patient has a minimum possible distance from an isocenter of the magnetic resonance device; and
positioning the patient table in the at least one second patient table position, wherein at least two degrees of freedom are available to the patient table for movement into the at least one second patient table position.

2. The method as claimed in claim 1, further comprising:
after positioning the patient table in the at least one second patient table position, adjusting at least one parameter of the region of interest to the at least one second patient table position.

3. The method as claimed in claim 1, further comprising:
after positioning the patient table in the at least one second patient table position, capturing second magnetic resonance data.

4. The method as claimed in claim 1, wherein four degrees of freedom are available to the patient table for movement of the patient table into the at least one second patient table position.

5. The method as claimed in claim 1, further comprising:
ascertaining the at least one second patient table position using a collision model.

6. The method as claimed in claim 5, wherein the collision model comprises an anatomy and/or size of the patient to be examined and/or position information for the patient table.

7. The method as claimed in claim 1, further comprising:
after positioning of the patient table in the at least one second patient table position, performing at least one adjustment step.

8. The method as claimed in claim 1, further comprising:
if there are two or more target anatomies of the patient, and thus two or more regions of interest, calculating and/or ascertaining a second patient table position for each of the regions of interest.

9. The method as claimed in claim 8, further comprising:
repeating the capturing of the first magnetic resonance data if not all target anatomies and/or regions of interest of the patient are captured and/or determined during a first capturing of first magnetic resonance data.

10. The method as claimed in claim 8, further comprising:
for ascertaining a plurality of second patient table positions, in each case taking a preceding second patient table position into account as a starting position for a patient table movement.

11. The method as claimed in claim 1, further comprising:
performing the capturing of the first magnetic resonance data with distortion correction.

12. A magnetic resonance device, comprising:
a magnet unit;
a patient receiving area at least partially surrounded by the magnet unit;
a patient table that is movable within the patient receiving area, and has at least two degrees of freedom with respect to its movement within the patient receiving area; and
a control unit embodied to control the magnetic resonance device such that a method for patient positioning using a patient table that is movable within a patient receiving area is executed as claimed in claim 1.

13. The magnetic resonance device as claimed in claim 12, wherein the patient table has four degrees of freedom for movement within the patient receiving area.

14. A non-transitory computer program product, which comprises a program and is loadable directly into a memory of a programmable control unit, with a program operable to control a method for patient positioning using a patient table that is movable in at least two spatial directions within a patient receiving area as claimed in claim 1, when the program is executed in the control unit.
